(19) United States
(12) Patent Application Publication
(10) Pub. No.: US 2023/0242968 A1
(43) Pub. Date: Aug. 3, 2023

(54) KITS AND METHODS USING COMBINED SAMPLES TO IMPROVE SENSITIVITY

(71) Applicant: 3M Innovative Properties Company, St. Paul, MN (US)

(72) Inventor: Michael Wohl, Bethesda, MD (US)

(21) Appl. No.: 18/015,885

(22) PCT Filed: Jul. 13, 2021
(86) PCT No.: PCT/US2021/041485

§ 371 Date: Jan. 12, 2023

Related U.S. Application Data
(60) Provisional application No. 63/051,116, filed on Jul. 13, 2020.

(51) Int. Cl. C12Q 1/6806; C12Q 1/686; C12Q 1/689

(52) U.S. Cl. CPC C12Q 1/6806; C12Q 1/686; C12Q 1/689

(57) ABSTRACT

Disclosed are methods for detecting analytes using combined sample collection types. In particular, a first sample is combined with a bio-aerosol sample. Also disclosed are kits that include components for collecting and combining a first sample with a bio-aerosol sample.

### CROSS-REFERENCE TO RELATED APPLICATIONS

This application claims priority to and the benefit of U.S. Provisional Patent Application Ser. No. 63/051,116, filed on Jul. 13, 2020, the disclosure of which is hereby incorporated by reference in its entirety.

### BACKGROUND OF THE DISCLOSURE

The present disclosure generally relates to medicine. More particularly, the present disclosure is directed to methods for detecting analytes using combined swab sample collection and bio-aerosol sample collection methods. The present disclosure is also directed to kits for detecting analytes using combined swab sample collection and bio-aerosol sample collection methods.

Clinical and laboratory testing is used to detect an analyte in a sample. Clinical testing is often used to determine infection and disease. Accurate testing is important to prevent transmission and spreading. Often detection of pathogen infection follows a “window period” after infection but before an infected individual exhibits symptoms. Many analytical tests lack the sensitivity and/or sensitivity during the window period and can produce false-negative results. When an individual tests negative, they may be cleared to return to work. A negative test can also cause the individual to reduce or remove protective measures such as mask wearing and social distancing. Thus, false negative results can result in transmission when prevention could occur. The ability to avoid false negative results from tests done early in the course of infection can reduce transmission. Suboptimal sample collection can decrease the overall sensitivity of testing and produce false negative test results.

Clinical and laboratory testing requires collection of a sample from a patient. A variety of samples can be collected from a patient and specific sample collection methods exist to collect the different sample types. These include, for example, tissue (biopsy), blood, saliva, sputum, nasopharyngeal swab, oropharyngeal (throat) swabs, throat washing, spinal fluid, and urine. For respiratory pathogen testing, nasopharyngeal swabbing is the sample collection method of choice. Oropharyngeal swabs are another acceptable method for collection for respiratory pathogen testing. Notably, the Center for Disease Control (CDC) recommends combining nasopharyngeal and oropharyngeal swabs in a single tube if both are collected. But, collection of both nasopharyngeal and oropharyngeal swabs is uncomfortable for patients and can pose a risk to healthcare workers. Others have paired nasopharangeal samples with saliva samples for detecting COVID-19 (see, Jamal et al. Clin Infect Dis. 2021 Mar. 15; 72 (6): 1064-1066; published online 2020 Jun. 25). Torretta et al. provides a list of all the currently available diagnostic techniques reported for COVID-19 testing (see, Torretta et al. Ear Nose Throat J. 2021 April; 100 (2_suppl):131S-138S; published online 2020 Aug. 31). While the prior art discloses combining, pairing, and comparing samples, each of the sample types are independently tested.

Collected sample and collection methods can suffer from inadequate amounts of the analyte to be detected in the sample. This can result in undetectable amounts of the analyte sought to be detected. Inadequate amounts of collected sample can also lead to reporting of false-negative results where a patient has the analyte but the sample does not contain enough of the analyte to be detected. To remedy these issues, samples are often further processed following collection to concentrate the amount of analyte in the portion of the sample that is tested.

One method to obtain detectable levels of the analyte to be detected is to collect more sample. This can be inconvenient or not possible in certain cases. Sample processing can also be performed to concentrate the analyte to be detected. Another method is to attempt to increase the sensitivity of the test such as performing longer test reagent incubation times, increasing the concentration of detection reagents, and performing more and longer cycles. Sample processing adds significant time required to conduct tests and to obtain test results. Requiring more reagents increases the cost of testing.

Accordingly, there exists a need for methods to improve detection of analytes in samples. The present disclosure accomplishes this by combining samples collected using different sample collection methods.

### BRIEF DESCRIPTION OF THE DISCLOSURE

The present disclosure generally relates to medicine. More particularly, the present disclosure is directed to methods for detecting pathogens using combined sample collection types.

In one aspect, the method includes collecting a first sample; collecting a bio-aerosol sample; introducing the first sample into a buffer, wherein the buffer elutes at least a portion of the first sample into the buffer to form a first eluent buffer; contacting at least a portion of the first eluent buffer with the bio-aerosol sample, wherein the first eluent buffer elutes at least a portion of the bio-aerosol sample to form a combined sample; and analyzing the combined sample for the analyte.

In another aspect, the present disclosure is directed to a kit, the kit comprising: at least one of a swab, a saliva collection vial, a sputum collection vial, and lavage collection components; a bio-aerosol collection device; a buffer; and instructions for using the kit.

### BRIEF DESCRIPTION OF THE DRAWINGS

FIGS. 1A-1C are illustrations depicting 3 combinations of samples. FIG. 1A depicts samples eluted separately and combined into a separate vial. FIG. 1B depicts samples eluted into same vial. FIG. 1C depicts samples eluted serially.

FIG. 2 is a graph depicting false negative swab PCR rate versus transmissibility of COVID. Viral transmission data and test false negative rate data both suggest that SARS-CoV-2 is undetectable until ˜2 days prior to symptom onset. Shown in cyan is the viral load data by day from onset of symptoms from He et al. (Nat. Med., 2020, 26:672-675). Shown in magenta is the false negative rate of tests by day from Kucirka et al. (Ann. Intern. Med., 2020). Transmission probability begins increasing ˜2 days before symptom onset, at the same time that the false negative rate of tests begin dropping. (From Jarvis and Kelley, Scientific Reports, 2021, 11:9221).

FIG. 3 is a graph combining swab and bio-aerosol relative strength from days of exposure. Swab and bio-aerosol samples independently normalized and combined signal non-normalized.

FIG. 4 is a Venn diagram depicting sample sensitivity, testing method agnostic (LFA or PCR) dynamic model based on pathogen temporal kinetics.

FIG. 5 is an illustration depicting the combination of sample types to increase sample sensitivity for PCR and LFA to reduce impacts of temporal pathogen kinetics and collection issues.

FIG. 6 is a table summarizing expected results of clinical tests comparing nasopharyngeal swab (NP) testing alone, bio-aerosol testing alone, and combined NP and bio-aerosol testing.

FIG. 7 is a flow diagram for bio-aerosol collection and detection.

FIG. 8 is an illustration depicting components of an exemplary embodiment of a test kit.

FIG. 9 is an illustration depicting swab collection in combination with mask insert (bio-aerosol) collection methods. In steps 1 and 2, a mask with an attached mask insert is worn by a subject. A collection substrate from the mask insert is removed from the mask and placed in a vial with elution/extraction buffer to elute/extract analyte in the bio-aerosol sample collected by the collection substrate of the mask insert (step 3). A swab is used to collect a swab sample from the subject (step 4). The swab is then placed in the same vial with the collection substrate of the mask insert (step 5) to elute/extract analyte from the swab. A portion of the combined sample is then analyzed (LFA shown) (step 6) and results are read (step 7).

FIG. 10 is an illustration depicting an exemplary mask insert for bio-aerosol sample collection. Inner collection substrate length 4.25 cm; width 1.25 cm. Outer substrates (front & back) length 6.3 cm, width 2.3 cm. Bio-aerosols pass through outer substrate to inner collection substrate. Inner collection substrate collects bio-aerosols and pathogens larger than 50 nanometers. COVID-19 approximately 100 nm; Influenza approximately 80-120 nm, Mycobacterium <7 microns, bio-aerosols containing respiratory pathogens mostly <10 microns.

FIG. 11 is an illustrating depicting removal of outer layers to expose collection substrate of mask insert embodiment.

FIG. 12 is an illustration depicting mask insert characteristics (adapted from Leung et al. Nat. Med. 26, 676-680 (2020)).

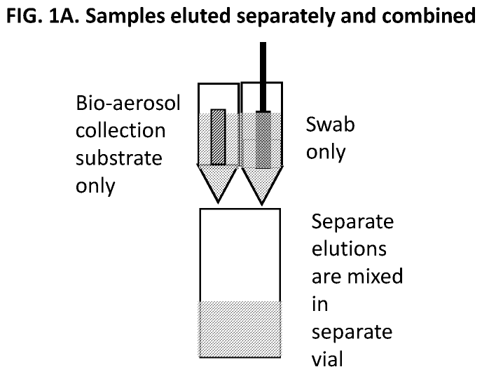

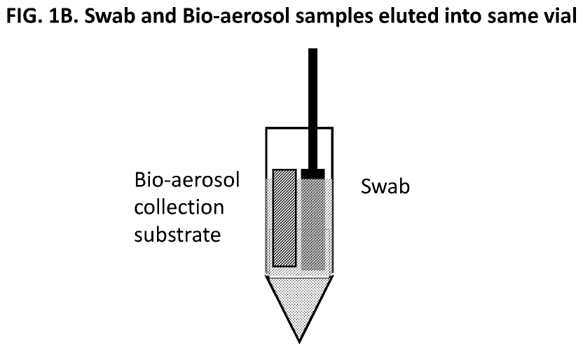

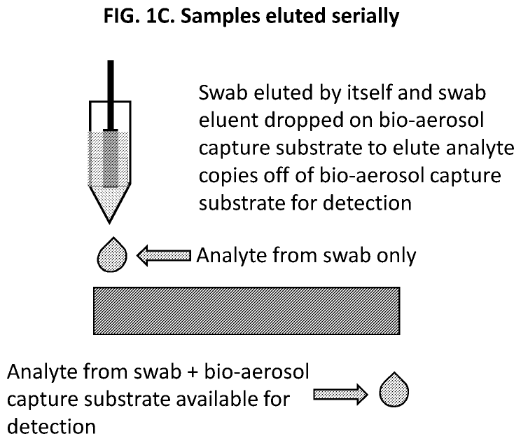

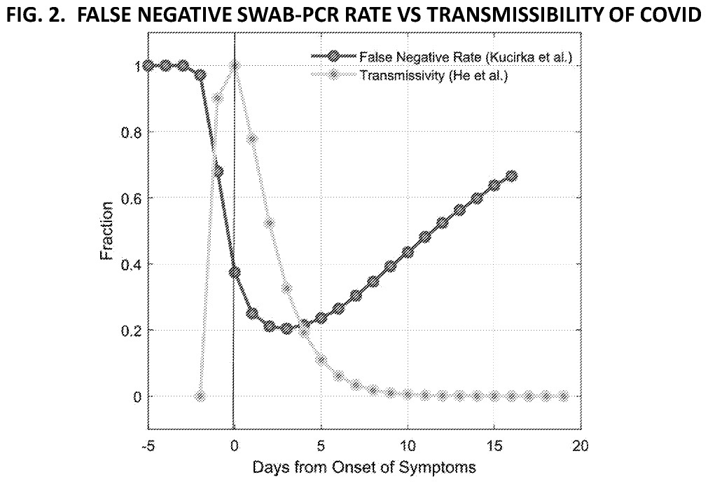

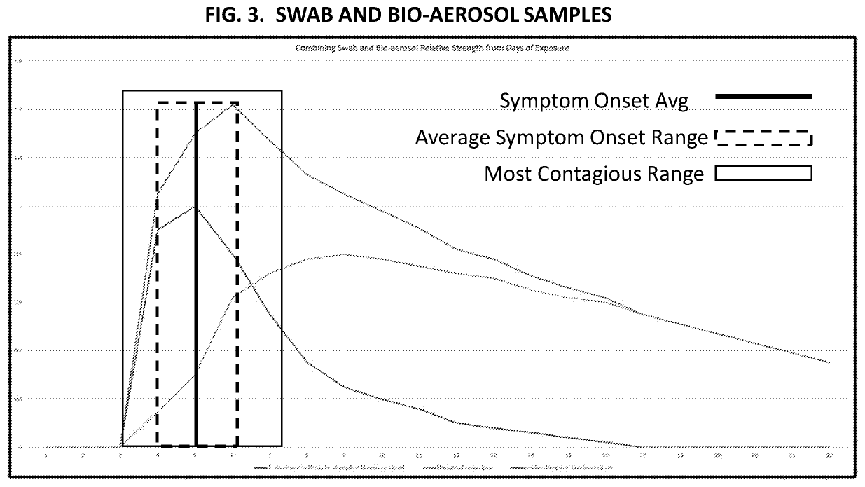

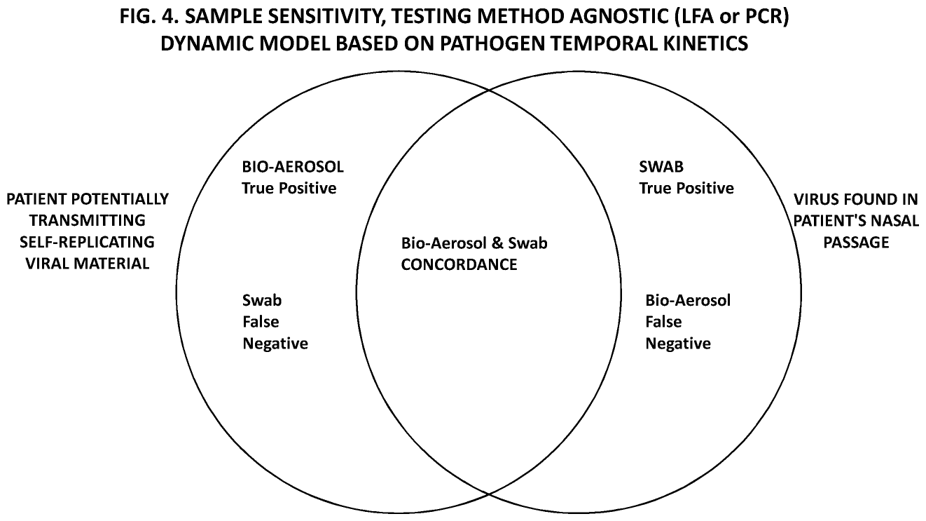

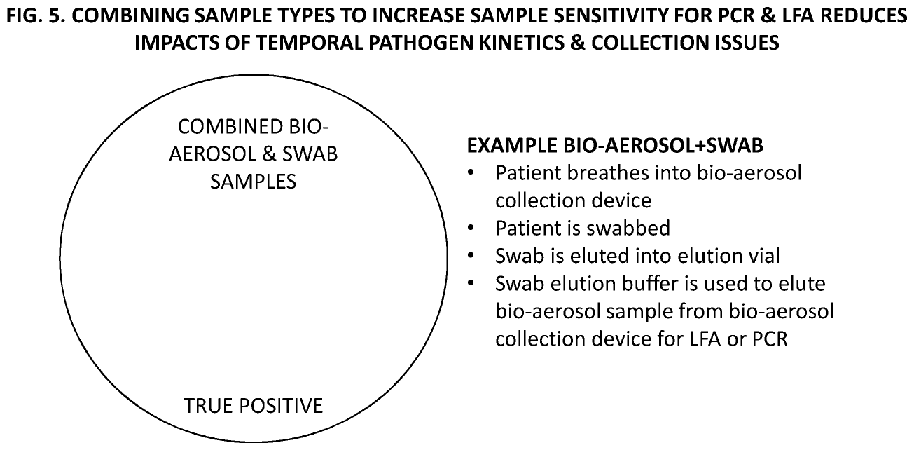

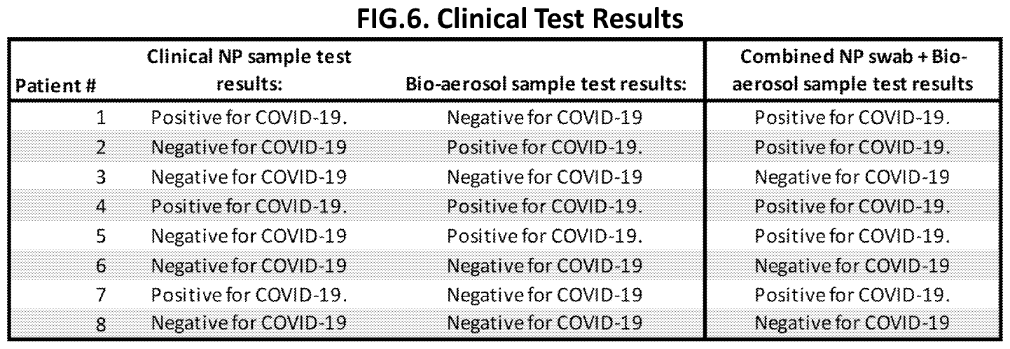

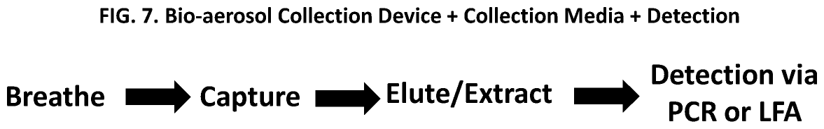

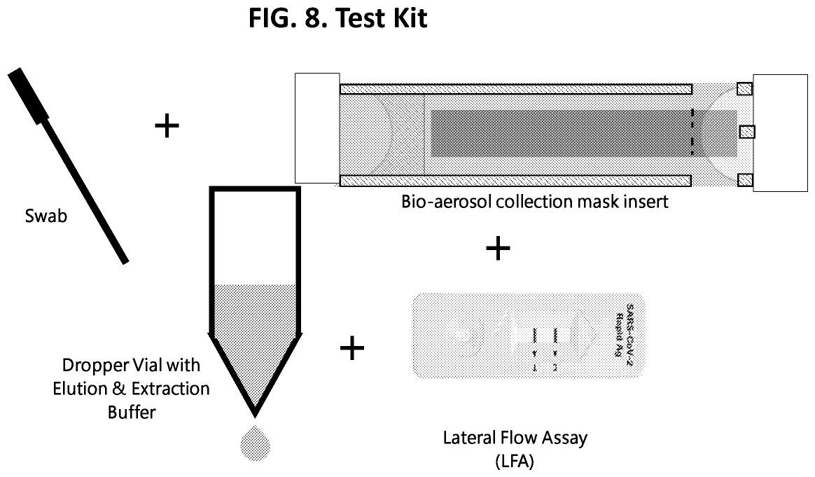

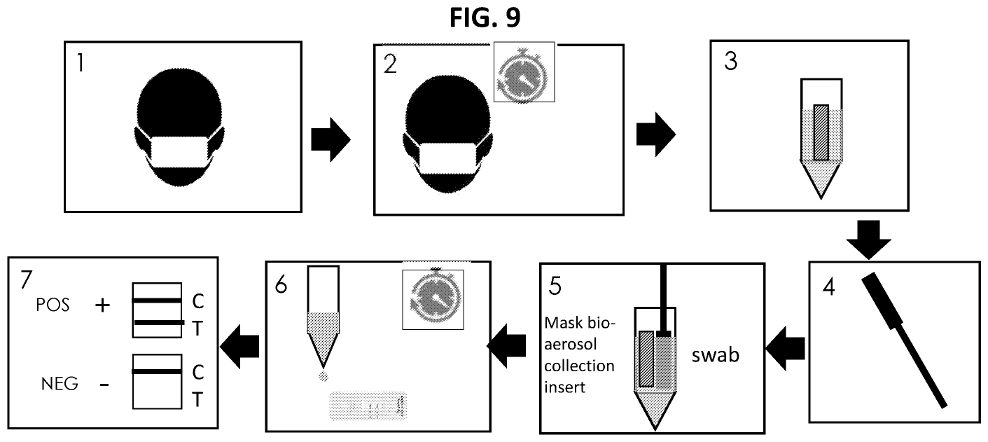

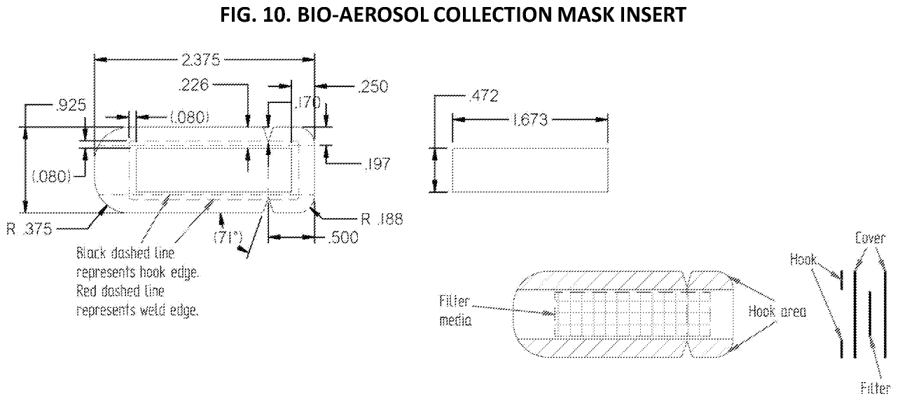

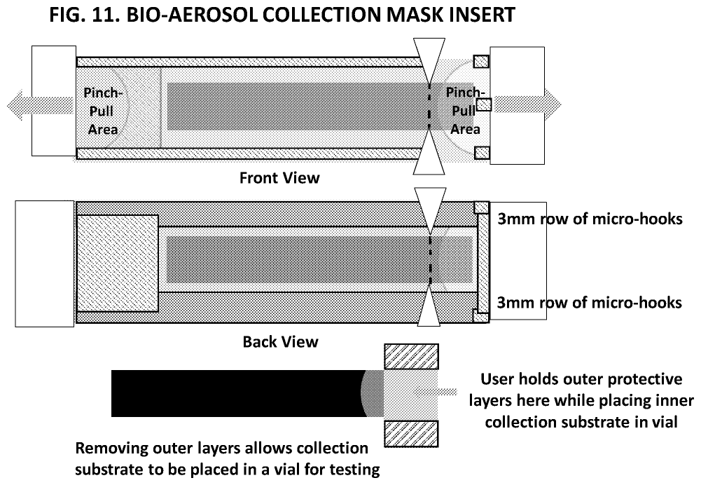

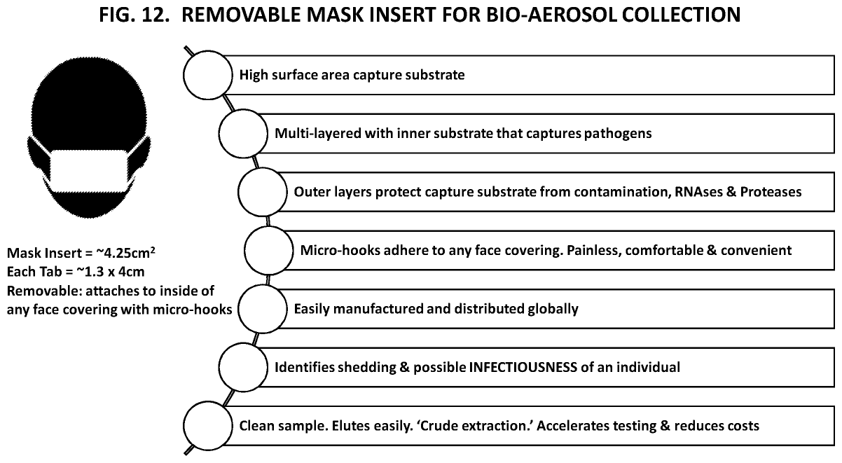

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Disclosed are kits and methods for detecting analytes using combined samples. In particular, the kits and methods combine a first sample obtained from a subject with a bio-aerosol sample obtained from the subject. The samples can be collected from the subject by a medical professional or by the subject with or without the aid of a medical professional. The first sample is eluted and the eluent is then used to elute the contents of the bio-aerosol sample collection device to form a combined sample. The combined sample is then analyzed.

The first sample is selected from a swab sample, a saliva sample, a sputum sample, an aspirate sample, a lavage sample, and combinations thereof. The swab sample includes a nasal swab, a nasopharyngeal swab, an oropharyngeal swab, a throat swab, a mid-turbinate swab, and combinations thereof. The lavage includes nasal lavage, oral lavage, nasopharyngeal lavage, oropharyngeal lavage, sinus lavage, tracheal lavage, bronchoalveolar lavage, lung lavage, and combinations thereof. The aspirate sample can be a nasal aspirate, a nasopharyngeal aspirate, an oropharyngeal aspirate, a throat aspirate, a tracheal aspirate, and combinations thereof.

In one embodiment, the bio-aerosol sample is collected using a bio-aerosol collection device into which the subject directs air (by blowing, breathing, humming, singing, speaking, and combinations thereof). The air enters the bio-aerosol sample collection device through an inlet. Air travels to a collection substrate that is positioned within the bio-aerosol collection device where analytes contained in the air sample are captured. The bio-aerosol collection device also includes an outlet that allow air to pass out of the bio-aerosol collection device after passing through the collection substrate. After collection and elution/extraction of the first sample. The eluent from the first sample is then introduced into the bio-aerosol sample collection device wherein analyte captured by the collection substrate of the bio-aerosol sample collection device is eluted to form a combined sample. The combined sample that contains analyte from the first sample and analyte from the bio-aerosol collection device sample is then analyzed. Suitable analysis methods are described herein such as amplification (polymerase chain reaction), immunoassay detection, mass spectrometry, and combinations thereof. In another embodiment, the collection substrate of the bio-aerosol collection device is placed in a vial containing an elution/extraction buffer and then the first sample is placed in the same vial with the bio-aerosol collection substrate wherein both the bio-aerosol collection substrate and the first sample are eluted/extracted together in the same vial. Following extraction/elution, a portion of the combined sample is analyzed.

In one embodiment, the bio-aerosol sample is collected using a mask or mask insert, wherein the subject wears a mask for a sufficient period of time such that an analyte contained in air expelled by the subject (via normal breathing, forced breathing, coughing, speaking, sneezing, and combinations thereof) contacts the mask insert and is captured by the mask insert. After collection of a first sample, the first sample is placed in an elution buffer to elute/extract analyte contained in the first sample. The eluent from the first sample is then used to elute analyte collected by a capture substrate of the mask insert wherein analyte captured by the collection substrate of the mask insert is eluted to form a combined sample with the first sample. The combined sample that contains analyte from the first sample and analyte from the mask insert sample is then analyzed. Suitable analysis methods are described herein such as amplification (polymerase chain reaction), immunoassay detection, mass spectrometry, and combinations thereof.

In one embodiment, the bio-aerosol sample is collected using a bio-aerosol collection device into which the subject directs an air sample. It should be understood that the bio-aerosol collection sample allows the subject to blow into the bio-aerosol collection device, cough into the bio-aerosol collection device, hum into the bio-aerosol collection device, speak into the bio-aerosol collection device, sing into the bio-aerosol collection device, and other methods wherein the subject can direct a breath sample toward the inlet of a bio-aerosol collection device. In particular, the subject directs air into an inlet of the bio-aerosol collection device. The bio-aerosol collection device is configured to direct the expired air sample to a collection medium (or substrate, also referred to herein as a “capture substrate”) contained in the bio-aerosol collection device wherein the collection substrate traps, attracts, and collects analytes contained in the expired breath of the subject. The bio-aerosol collection device is also configured to allow the air sample to flow through an outlet to avoid a pressure differential that could dislodge and/or affect the positioning of the collection substrate. In one embodiment, the bio-aerosol sample is collected using a bio-aerosol collection device that includes a hollow housing comprising an inlet; a capture substrate; and an outlet. The collection substrate is positioned in the bio-aerosol collection device such that the subject's bio-aerosol sample flows through, over, and/or toward the collection substrate wherein an analyte contained in the bio-aerosol sample is captured by the collection substrate. The collection substrate can also be removed from the bio-aerosol collection device. Removing the collection substrate allows the other components of the bio-aerosol collection device to be reused. If the bio-aerosol collection device is to be reused, the device is cleaned and sterilized and a new collection substrate is placed into the bio-aerosol collection device. Removing the collection substrate also allows for the collection substrate to be placed in a vial for storage, transport, processing, testing, and combinations thereof. To avoid or minimize contamination of the collection substrate when transferring the collection substrate to the vial, a tool such as a swab, forceps and/or a probe can be used to dislodge the collection substrate from the bio-aerosol collection device and into the vial. A swab that is used to collect the swab sample can be used to transfer the collection substrate from the bio-aerosol collection device into a vial and then the swab can be placed in the same vial with the collection substrate. Alternatively, the swab that is used to collect the swab sample can be used to transfer the collection substrate from the bio-aerosol collection device into a vial and then the swab is placed in a different (second) vial. Additionally, or alternatively, the bio-aerosol collection device can be configured to release the collection substrate from its location within the bio-aerosol collection device without any tools or with minimal use of a tool. For example, the bio-aerosol collection device can include a “locking mechanism” such that when the device arrives to the clinician, the collection substrate is “locked” into the bio-aerosol collection device. After the subject provides the bio-aerosol sample to the bio-aerosol collection device, the clinician performs an action (such has a twisting motion, pulling motion, and the like) on the device to release the collection substrate from the bio-aerosol collection device and allow the collection substrate to be transferred from the bio-aerosol collection device. As described herein, the vial can include a solution (transport medium, storage medium, elution buffer, extraction buffer, and combinations thereof) or be empty. The vial could also include a desiccant.

In some embodiments, the bio-aerosol sample is collected using an expiratory droplet collection device. Suitable methods for collecting expiratory droplet samples include droplet collection devices such as, for example, masks and mask inserts.

The capture substrate of the bio-aerosol collection device and the expiratory droplet collection device (e.g., mask insert) is suitably made with synthetic fibers, natural fibers, and combinations thereof. Fibers used to form the capture substrate include hydrophobic fibers, hydrophilic fibers, and combinations thereof. Hydrophobic fibers include, for example, polylactones, poly(caprolactone), poly (L-lactic acid), poly (glycolic acid), similar co-polymers poly(alkyl acrylate), polybutadiene, polyethylene, polystyrene, polyacrylonitrile, polyethylene (terephthalate), polysulfone, polycarbonate, poly(vinyl chloride), and combinations thereof. Hydrophilic fibers include, for example, linear poly(ethylenimine), cellulose, cellulose acetate and other grafted cellulosics, poly (hydroxyethylmethacrylate), poly (ethyleneoxide), polyvinylpyrrolidone, poly(acrylic acid), poly(ethylene glycol), poly(vinyl alcohol), poly (vinyl acetate), poly(acrylamide), proteins, poly (vinyl pyrrolidone), poly(styrene sulfonate), and combinations thereof. Other suitable fiber materials include, for example, acrylonitrile/butadiene copolymer, cellulose, cellulose acetate, chitosan, collagen, DNA, fibrinogen, fibronectin, nylon, poly(acrylic acid), poly(chloro styrene), poly(dimethyl siloxane), poly(ether imide), poly(ether sulfone), poly(ethyl acrylate), poly(ethyl vinyl acetate), poly(ethyl-co-vinyl acetate), poly(ethylene oxide), poly(ethylene terephthalate), poly(lactic acid-co-glycolic acid), poly(methacrylic acid) salt, poly(methyl methacrylate), poly(methyl styrene), poly(styrene sulfonic acid) salt, poly(styrene sulfonyl fluoride), poly(styrene-co-acrylonitrile), poly(styrene-co-butadiene), poly(styrene-co-divinyl benzene), poly(vinyl acetate), poly(vinyl alcohol), poly(vinyl chloride), poly(vinylidene fluoride), polyacrylamide, polyacrylonitrile, polyamic acid (PAA), polyamide, polyaniline, polybenzimidazole, polycaprolactone, polycarbonate, polydimethylsiloxane-co-polyethyleneoxide, polyetheretherketone, polyethylene, polyethyleneimine, polyimide, polyisoprene, polylactide, polypropylene, polystyrene, polysulfone, polyurethane, polyvinylpyrrolidone, proteins, SEBS copolymer, silk, and styrene/isoprene copolymer. Polymer blends such as, for example, poly(vinylidene fluoride)-blend-poly(methyl methacrylate), polystyrene-blend-poly(vinylmethylether), poly(methyl methacrylate)-blend-poly(ethyleneoxide), poly(hydroxypropylmethacrylate)-blend poly(vinylpyrrolidone), poly(hydroxybutyrate)-blend-poly(ethylene oxide), protein blend-polyethyleneoxide, polylactide-blend-polyvinylpyrrolidone, polystyrene-blend-polyester, polyester-blend-poly(hyroxyethyl methacrylate), poly(ethylene oxide)-blend poly(methyl methacrylate), poly(hydroxystyrene)-blend-poly(ethylene oxide). The fiber materials used to form the capture substrate can be selected to cause the analyte (including a carrier containing the analyte) to travel from one portion of the capture substrate to another portion of the capture substrate. The fiber materials used to form the capture substrate can be selected to cause the analyte (including a carrier containing the analyte) to travel from one portion of the capture substrate out of the capture substrate to an analytical substrate and/or a collection vial.

Another suitable capture substrate can be electret (including thermoelectrets and fibrillated electret film). Particularly suitable electret includes polypropylene and polylactic acid. Electret is a dielectric material having a quasi-permanent electric charge or dipole polarization. Electret can be obtained from commercially available sources. Electret can be prepared by heating and simultaneously exposing a material to an electric field, whereby many dipoles in the material become oriented in a preferred direction. After the heating, the material is “frozen” and is able to keep the position of its electric dipoles for a long period of time. Suitable materials for preparing electrets include, for example, materials can now be used to fabricate thermoelectrets, including organic materials such as ebonite, naphthalene, polymethyl-methacrylate, and many polymers, and inorganic materials such as sulfur, quartz, glasses, steatite, and some ceramics. Electret fibrous membranes are particularly suitable. Polyvinylidene fluoride (PVDF)/polytetrafluoroethylene (PTFE) NP electret nanofiber membranes can be formed by electrospinning. Fibrillated electret film disclosed in van Turnhout (U.S. Pat. No. 3,998,916) is also suitable. One collection method is disclosed in PCT/US2007/061082 to Kanzer Kanzer discloses a filtering face mask having a biosampling material affixed to the mask that entraps pathogens expired by the wearer. Another suitable mask is disclosed in U.S. Pat. No. 6,119,691 to Angadjivand. Angadjivand discloses a mask having an electret filter material.

The collection substrate can be an immunochromatographic test substrate, a colloidal gold test substrate, and combinations thereof. The substrate can include, for example, a quantum dot-marked test substrate, a colloidal gold-marked test substrate, a colloidal selenium-marked test substrate, an upconversion phosphorescence-marked test substrate, a nano rare earth fluorescent complex-marked test substrate, a temporal resolution chromatography test substrate, a chemiluminescence test substrate, and other test substrates.

The collection substrate can also be a multi-layer substrate. The multi-layer collection substrate has an inner collection substrate that is protected on all sides by additional layers. The inner collection layer is made of a material having high surface area for collecting the analyte to be detected. The inner collection substrate can also be released and/or separated from protective layers. The protective layers and inner collection substrate can be made of different materials designed for the specific purposes of the layer (e.g., protection and sample collection). For example, the inner collection substrate can be of a corrugated, double-sided polyester swab material. The inner collection substrate can be coated with a reagent to retain the analyte load collection. The inner collection substrate and/or protective layer(s) can also be coated with a reagent to stabilize the analyte. The protective layer can be an air-penetrating, touch-protective coating.

The methods of the present disclosure combine samples obtained using a swab with a bio-aerosol collection device. In another embodiment, the methods of the present disclosure can combine a swab sample and a bio-aerosol sample with a third sample collection method. Suitable third samples include, for example, saliva, sputum, nasal lavage, oral lavage, oral swab, oropharyngeal swab, bronchoalveolar lavage, blood, urine, stool, and combinations thereof. The method includes obtaining a swab sample, obtaining a bio-aerosol sample, obtaining a third sample, eluting the swab sample to form a swab eluent, using at least a portion of the swab eluent to elute bio-aerosol sample to form a combined eluent, analyzing the combined eluent, and analyzing the third sample.

In another embodiment, the combined swab sample eluent and bio-aerosol sample eluent (also referred to herein as the “combined sample”) is placed in a sterile transport container. The combined swab sample eluent and bio-aerosol sample eluent can then be stored and/or transported to be analyzed at a laboratory test facility. Following collection, the sample can be placed in a sterile transport container containing a transport medium. Suitable transport media include, for example, viral transport medium (VTM), universal transport material (UTM), Amies transport medium, and sterile saline. The transport media can include, for example, RNase inhibitors, DNase inhibitors, protease inhibitors, preservatives, stabilizers, antimicrobial additives such as silver-containing antimicrobial agents and antimicrobial polypeptides, analgesic compounds such as lidocaine, antibiotics such as neomycin, thrombogenic compounds, nitric oxide releasing compounds such as sydnonimines and NO-complexes, bacteriocidal compounds, fungicidal compounds, bacteriostatic compounds, other pharmaceutical compounds, adhesives, fragrances, odor absorbing compounds, preservatives, and nucleic acids, including deoxyribonucleic acid, ribonucleic acid, and nucleotide analogs.

In another embodiment, the swab is placed into a first vial and the bio-aerosol collection substrate is placed into a second vial. In another embodiment, the swab and the bio-aerosol collection substrate are placed into the same vial. The vial in which the swab and/or bio-aerosol collection substrate is placed can further contain a transport medium. Suitable transport media include saline, phosphate-buffered saline, HEPES, tris-buffered saline, water, commercially available transport media (such as UTM viral transport medium commercially available from COPAN Diagnostics, (Murrietta, Calif.)), and combinations thereof.

Particularly suitable elution buffers include nonionic surfactants. Suitable a surfactants include TRITON X100, TRITON X100 reduced, TRITON X114, TRITON X45, Nereid, polyoxyethylene lauryl ether, sodium dodecyl sulfate (SDS), sodium lauryl sulfate (SLS), NP-40, polysorbates, CHAPS (3-[(3 -cholamidopropyl)dimethylammonio]-1-propanesulfonate), DOC, cetyl trimethylammonium bromide (CTAB), Brij 52, and combinations thereof. The elution buffer can be a solution of about 0.25% saline to about 0.9% saline including the surfactant. The elution buffer can further include a pH buffer, salts, chelators, and combinations thereof. As used herein, the “elute” and “extract” are used to describe removing at least a portion of analytes from the sample. For example, when the sample is collected using a swab, the elution buffer removes at least a portion of the sample from the swab. The elution buffer can also disrupt the analyte in a manner to allow components of the analyte to be detected. For example, the elution buffer can cause the membrane of a microorganism to be disrupted to allow the microorganism's DNA or RNA to be transferred into the buffer and then the DNA and/or RNA of the microorganism can be detected to establish presence of the microorganism in the sample obtained from the subject.

Any method for analyzing the samples of the present disclosure that are known in the art are suitable for analyzing the combined samples. Suitable methods include, for example, DNA and RNA amplification methods (e.g., PCR, RT-PCR, qPCR, loop-mediated isothermal amplification (LAMP)), immunoassay detection methods such as, for example, Lateral Flow assays (Rapid lateral flow), Vertical flow assays, optical immunoassay method (e.g., Biostar, Inc., Boulder, Colo.), Western blot analysis, direct fluorescent antibody assay, antigen enzyme immunoassay, and enzyme linked immunosorbent analysis (ELISA), chromatography such as, for example, high pressure liquid chromatography (HPLC), gas chromatography, capillaripheresis, 2D and 3D gel electrophoresis, mass spectrometry, and combinations thereof.

The combined sample can be analyzed with devices such as lateral flow assay and vertical flow assay. Lateral Flow and Vertical Flow Assays use strips that contain one or more capture zones: a control line that detects the presence of all antibodies in the sample and a test line that specifically reacts with an analyte to be detected.

In another embodiment, the combined sample can be analyzed using an antigen test. As known in the art, an antigen test contains antibodies on the test device that will bind to the antigen contained in the sample. In another embodiment, the combined sample can be analyzed with an antibody test. As known in the art, an antibody test contains antigens on the test device that will bind to the antibodies contained in the sample. In both antigen and antibody tests, binding of the antibody and antigen triggers a visible result indicating that the subject is infected.

Analysis can be automated using commercially available platforms such as Cobas Amplicor (Roche Molecular Diagnostics, Pleasanton, Calif.).

Once placed in a transport container, the collected and combined samples can be stored until later analysis or processed for analysis. Processing can include lysis, extraction, and other known processing steps to detect the pathogen from the collected samples.

Analysis can use point of care platforms and/or offsite high throughput platforms.

Analytes include microorganisms, biological molecules, and chemical molecules. Particularly suitable microorganisms to be detected are pathogens. As used herein, “pathogen” refers to microorganisms such as, for example, bacteria, fungi, and viruses. The term “pathogen” also refers to viroids, prions, and proteins.

Suitable analytes are contained in gases and aerosol droplets in air expelled by the user. Suitable analytes include microorganisms, chemicals, proteins, nucleic acids, and combinations thereof. Suitable microorganisms include bacteria and viruses.

Particularly suitable microorganisms include pathogens. The term “pathogen” is used according to its ordinary meaning to refer to bacteria, viruses, and other microorganisms that directly or indirectly cause disease. Exemplary pathogens include, for example, Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Eberthella, Francisella, Haemophilus, Bacteroides, Listeria, Erysipelothrix, Acinetobacter, Brucella, Pasteurella, Flavobacterium, Fusobacterium, Streptobacillus, Calymmatobacterium, Legionella, Treponema, Borrelia, Leptospira, Actinomyces, Nocardia, Rickettsia, Micrococcus, Mycobacterium, Neisseria, Campylobacter, pathogenic viruses such as, for example, Papilloma viruses, Parvoviruses, Adenoviruses, Herpesviruses, Vaccine virus, Arenaviruses, Coronaviruses, Rhinoviruses, Respiratory syncytial viruses, Influenza viruses, Picornaviruses, Paramyxoviruses, Reoviruses, Retroviruses, Rhabdoviruses, human immunodeficiency virus (HIV), Taenia, Hymenolepsis, Diphyllobothrium, Echinococcus, Fasciolopsis, Heterophyes, Metagonimus, Clonorchis, Fasciola, Paragonimus, Schistosoma, Enterobius, Trichuris, Ascaris, Ancylostoma, Necator, Wuchereria, Brugi, Loa, Onchocerca, Dracunculus, Naegleria, Acanthamoeba, Plasmodium, Trypanosoma, Leishmania, Toxoplasma, Entamoeba, Giardia, Isospora, Cryptosporidium, Enterocytozoa, Strongyloides, Trichinella, a fungus causing, for example, Ringworm, Histoplasmosis, Blastomycosis, Aspergillosis, Cryptococcosis, Sporotrichosis, Coccidiodomycosis, Paracoccidioidomycosis, Mucomycosis, Candidiasis, Dermatophytosis, Protothecosis, Pityriasis, Mycetoma, Paracoccidiodomycosis, Phaeohphomycosis, Pseudallescheriasis, Trichosporosis, Pneumocystis, Adenovirus, Coronavirus (Coronavirus 229E, Coronavirus HKU1, Coronavirus NL63, Cornavirus OCL43), Human Metapneumovirus, Human Rhinovirus, Enterovirus, Influenza A, Influenza B, Middle East Respiratory Syndrom Coronavirus (MERS-CoV), Parainfluenza Virus 1, Parainfluenza Virus 2, Parainfluenza Virus 3, Parainfluenza Virus 4, Respiratory Syncytial virus, Bordetella parapertussis, Bordetella pertussis, Chlamydia pneumonia, Mycoplasma pneumoniae, and combinations thereof.

Particularly suitable chemicals include ketones, nicotine, cocaine, opioids, marijuana, benzodiazepines, amphetamines, barbiturates, and combinations thereof.

Samples can also be analyzed for proteins, DNA, and RNA.

Samples can be simultaneously analyzed for multiple different analytes (e.g., multiplex assay). For example, the methods of the present disclosure are particularly suitable for detecting and differentiating RNA from SARS-CoV-2, influenza A virus, and influenza B virus in upper or lower respiratory samples in a multiplex assay.

### EXAMPLES

### Example 1

This Example outlines testing of different sample collection methods and combinations of sample collection methods.

1. Device: Mask Insert

Use Case: Essential Workers

Detection Via: multi-site sputum versus (oral+NP) Swab via PCR versus Bio-aerosol via PCR

Pathogens Tested: COVID+TB

Enrollment criteria: Presumed positive

Scientific Need: Test longer collection period and non-localized sampling to screen for COVID+TB to protect essential workers

Behaviors for Bio-aerosol collection: Wear standard surgical mask delivered with collection substrate. Speak, cough, breath as normal.

Collection time Minimum of 4 hours

Sample Handling: POC worker removes bio-aerosol sample collector from mask and places it in 5 mL Eppendorf. 1 mL of UTM. 10 sec mechanical vibration, spin for 30 to acquire sample in buffer.

2. Device: Mask Insert

Use Case: Essential Workers

Detection Via: Multi-site (Oral+NP) Swab via PCR vs Bio-aerosol via PCR

Pathogens Tested: Respiratory panel

Enrollment criteria: Cough of any duration or shortness of breath or sore throat

Scientific Need: Test longer collection period and non-localized sampling to screen for a panel of respiratory pathogens to protect essential workers

Behaviors for Bio-aerosol collection: Wear standard surgical mask delivered with collection substrate. Speak, cough, breathe as normal.

Collection time Minimum of 4 hours

Sample Handling: POC worker removes bio-aerosol sample collector from mask and places it in 5 mL Eppendorf. 1 mL of UTM. 10 sec mechanical vibration, spin for 30 to acquire sample in buffer.

3. Device: Blow Tube

Use Case: General population

Detection Via: Multi-site (Oral+NP) Swab via PCR vs Bio-aerosol via PCR vs Sputum

Pathogens Tested: COVID+TB (need to determine single elution protocol)

Enrollment criteria: Presumed positive

Scientific Need: Compare rapid bio-aerosol collection vs NP swab collection method, ideally for days −2 to +7 of symptom onset for COVID. Sputum for TB

Behaviors for Bio-aerosol collection: Deep Breaths×20, Cough×10, Count to 20.

Collection time: 10-15 minutes

Sample Handling: POC worker removes bio-aerosol sample collector and places it in 5 mL Eppendorf. 1 mL of UTM. 10 sec mechanical vibration, spin for 30 to acquire sample in buffer.

4. Device: Blow Tube

Use Case: General population

Detection Via: Multi-site (Oral+NP) Swab via PCR vs Bio-aerosol via PCR

Pathogens Tested: Respiratory panel

Enrollment criteria: Cough of any duration OR Shortness of breath OR Sore throat

Scientific Need: Compare rapid bio-aerosol collection vs NP swab collection method, ideally for days −2 to +7 of symptom onset for a panel of respiratory diseases

Behaviors for Bio-aerosol collection: Deep Breaths×20, Cough×10, Count to 20.

Collection time: 10-15 minutes

Sample Handling: POC worker removes bio-aerosol sample collector and places it in 5 mL Eppendorf. 1 mL of UTM. 10 sec mechanical vibration, spin for 30 to acquire sample in buffer.

5. Device: Blow Tube

Use Case: General population

Detection Via: Multi-site (Oral+NS) Swab+Bio-aerosol via PCR vs NP swab via PCR

Pathogens Tested: COVID+TB

Enrollment criteria: Presumed positive

Scientific Need: Compare combining rapid bio-aerosol collection with NP swab collection method for days −2 to +7 of symptom onset for COVID

Behaviors for Bio-aerosol collection: Deep Breaths×20, Cough×10, Count to 20

Collection time: 10-15 minutes

Sample Handling: POC worker removes bio-aerosol sample collector and places it in 5 mL Eppendorf. 1 mL of UTM. 10 sec mechanical vibration, spin for 30 to acquire sample in buffer.

6. Device: Blow Tube

Use Case: General population

Detection Via: Multi-site (Oral+NS) Swab+Bio-aerosol via PCR vs NP swab via PCR

Pathogens Tested: Respiratory panel

Enrollment criteria: Cough of any duration OR Shortness of breath Or Sore throat

Scientific Need: Compare combining rapid bio-aerosol sample with NP swab collection method, ideally for days −2 to +7 of symptom onset for Respiratory panel.

Behaviors for Bio-aerosol collection: Deep Breaths×20, Cough×10, Count to 20

Collection time: 10-15 minutes

Sample Handling: POC worker removes bio-aerosol sample collector and places it in 5 mL Eppendorf. 1 mL of UTM. 10 sec mechanical vibration, spin for 30 to acquire sample in buffer.

7. Device: Blow Tube

Use Case: General population

Detection Via: Multi-site (Oral+NP) Swab via PCR vs Bio-aerosol via Rapid Detection Test (RDT) (visual and with reader)

Pathogens Tested: COVID

Enrollment criteria: Presumed positive

Scientific Need: Compare qualitative testing via RDT of rapid bio-aerosol sample only vs quantitative NP swab collection tested via reference PCR, ideally for days −2 to +7 of symptom onset for Respiratory panel.

Behaviors for Bio-aerosol collection: Deep Breaths×20, Cough×10, Count to 20

Collection time: 10-15 minutes

Sample Handling: Patient performs behaviors. POC applied necessary elution/extraction buffer drops to bio-aerosol substrate.

8. Device: Blow Tube

Use Case: General population

Detection Via: Multi-site (Oral+NP) Swab via PCR vs NS swab+Bio-aerosol via RDT (visual and with reader)

Pathogens Tested: COVID

Enrollment criteria: Presumed positive

Scientific Need: Compare qualitative testing via RDT of rapid bio-aerosol sample combined with NS sample vs quantitative NP swab collection tested via reference PCR, ideally for days −2 to +7 of symptom onset for Respiratory panel.

Behaviors for Bio-aerosol collection: Deep Breaths×20, Cough×10, Count to 20

Collection time: 10-15 minutes

Sample Handling: Patient performs bio-aerosol collection behaviors in device. POC worker swabs patient with an NS swab and elutes in RDT elution/extraction buffer as normal. POC applies necessary elution/extraction buffer drops to bio-aerosol substrate to test via RDT.

While embodiments described herein include combining samples obtained using a bio-aerosol collection device with a sample collected using a swab collection method, it should be understood that the samples obtained using a bio-aerosol collection device can be analyzed alone (without combining with a second sample). Advantageously, the methods of the present disclosure make viral loads available in a density that can be used with a wide variety of tests such as amplification, antibody, antigen, and other paper-based tests. The methods of the present disclosure are particularly advantageous when a sample collected using a bio-aerosol collection device is combined with a swab sample because the combination of samples specifically increase viral load availability.

The methods of the present disclosure advantageously allow for overall sensitivity of testing and reducing false negative test results. In particular, combining an expiratory droplet sample with a swab sample can maximize test sensitivity and reduce the amounts of testing resources used to analyze the sample. Further, combining a bio-aerosol sample and a shed virus using a bio-aerosol collection device with other samples collected using a different collection methods advantageously augments viral loads collected that can be tested thereby increasing test sensitivity. Combining a bio-aerosol sample and a shed virus collected using a bio-aerosol collection device with a sample collected using a swab collection method can also advantageously smooth out any signal diminishment inherent to sampling from the upper respiratory tract versus a saliva sample, for example. Combining bio-aerosol collection device with a sample collected using a swab collection can also advantageously assist with downstream test handling. Combining bio-aerosol collection device with a sample collected using a swab collection can also advantageously reduce false negatives because pathogen loads collected using at least two different sample collection methods can be increased.

1. A method for detecting an analyte in a combined sample obtained from a subject, the method comprising:
collecting first sample;
collecting a bio-aerosol sample;
introducing the first sample into a buffer, wherein the buffer elutes at least a portion of the first sample into the buffer to form a first eluent buffer;
contacting at least a portion of the first eluent buffer with the bio-aerosol sample, wherein the first eluent buffer elutes at least a portion of the bio-aerosol sample to form a combined sample; and
analyzing the combined sample for the analyte.

2. The method of claim 1, wherein the bio-aerosol sample is collected using a droplet collection device.

3. The method of claim 2, wherein the droplet collection device is selected from a mask, a mask insert, an expiratory droplet collection strip, an electret filter material, and combinations thereof.

4. The method of claim 1, wherein the bio-aerosol collection is using a bio-aerosol collection device wherein the subject blows into the bio-aerosol collection device, coughs into the bio-aerosol collection device, hums into the bio-aerosol collection device, speaks into the bio-aerosol collection device, or a combination thereof.

5. The method of claim 2, wherein the bio-aerosol sample is collected using a material capable of entrapping a pathogen expired by the subject.

6. The method of claim 1, wherein the first sample is selected from a swab sample, a saliva sample, a sputum sample, an aspirate sample, a lavage sample, and combinations thereof.

7. The method of claim 1, wherein the analyzing is performed using amplification, an immunoassay, or a combination thereof.

8. The method of claim 7, wherein the analyzing comprises a polymerase chain reaction.

9. The method of claim 1, wherein the analyzing is performed using lateral flow analysis, vertical flow analysis, and combinations thereof.

10. The method of claim 1, wherein the combined sample is analyzed by introducing at least a portion of the combined sample into a lateral flow assay, a vertical flow assay, or a combination thereof.

11. The method of claim 1, wherein the analyte is a pathogen.

12. The method of claim 1, wherein the bio-aerosol collection device comprises a collection substrate and the collection substrate is transferred from the bio-aerosol collection device into a vial.

13. The method of claim 12, wherein the vial comprises a buffer.

14. The method of claim 12, wherein the vial comprises the first eluent buffer.

15. A kit comprising:
at least one of a swab, a saliva collection vial, a sputum collection vial, and lavage collection components;
a bio-aerosol collection device;
a buffer; and
instructions for using the kit.

16. The kit of claim 15, further comprising an analytical device.

17. The kit of claim 16, wherein the analytical device comprises a lateral flow assay, a vertical flow assay, or a combination thereof.

18. The kit of claim 15, wherein the bio-aerosol collection device is a mask insert.

19. The kit of claim 15, wherein the bio-aerosol collection device is a blow tube device.
